(19) United States
(12) Patent Application Publication
(10) Pub. No.: US 2010/0040562 A1
(43) Pub. Date: Feb. 18, 2010

(54) NOVEL COMPOSITION

(76) Inventors: Peter Frost, Surrey (GB); Hanna Maria Malmstrom, Surrey (GB); Mark Penegar, Surrey (GB); Alexander Thomas Platts, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/517,675

(22) PCT Filed: Dec. 6, 2007
(86) PCT No.: PCT/EP2007/063480

§ 371 Date: Jun. 4, 2009

(30) Foreign Application Priority Data
Dec. 8, 2006 (GB) 0624578.1

(51) Int. Cl. A61K 8/18; A61K 9/12; A61Q 11/00

(52) U.S. Cl. 424/47; 424/49

(57) ABSTRACT

A post-foaming dentifrice composition having a viscosity of at least 80,000 mPas comprising a combination of a first thickening agent which is xanthan gum, a second thickening agent which is carrageenan and/or a polyacrylic acid and a thickening silica, together with a post-foaming agent to expand the composition to a foam.

This invention relates to dentifrice compositions, in particular to a dentifrice composition that can be stored in a pressurised container from which it can be dispensed onto a toothbrush in the form of a post-foaming gel or paste.

WO-A-01/62212 (Henkel) describes a post-foaming dentifrice of relatively high viscosity (from 30,000 to 100,000 mPas), which can be extruded from a dispenser in the form of a ribbon of paste or gel with delayed foaming to give a mousse-like foam at the site of application. This is achieved by incorporating a “post-foaming agent” i.e. a liquefied propellant gas which boils at −15 to 35° C. into the dentifrice composition so that ambient or body temperature causes the propellant to vaporise.

US-A-2004/247534 (Stoltz) describes a self-foaming fluoride gel containing a hydrocarbon aerosol propellant.

WO-A-2006/52476 (Colgate) describes a post-foaming oral care mousse of relatively low viscosity (less than 30,000 mPas) containing a compressed liquid propellant.

These prior documents all indicate that such post-foaming dental compositions may be dispensed from so-called bag in can dispensers in which the substance containing the post-foaming agent is contained within a collapsible envelope within an enclosing can, with a pressurised propellant between the can and the envelope, the propellant squeezing the substance out of the envelope when a valve on the envelope is opened to the environment. Bag in can dispensers may be of the so-called bag on valve type in which the bag is attached to the valve, or of the so-called bag on can type in which the bag is attached to the can. Both types are included within the term bag in can as used herein. The reverse bag in can construction in which the pressurised propellant is in an expandable envelope within the enclosing can, with the substance containing the post-foaming agent in the space between the can and the envelope is also known. In this reverse construction, expansion of the propellant within the bag causes the expanding bag to squeeze the substance out of the space when a valve on the can is opened to the environment.

The present invention is concerned with a post-foaming dentifrice of high viscosity having a reduced tendency for stringiness and with good stand-up when dispensed onto a toothbrush from a bag in can dispenser. These advantages have been achieved by using as a thickening system a particular combination of thickening agents together with a thickening silica.

Accordingly the present invention provides a post-foaming dentifrice composition having a viscosity of at least 80,000 mPas comprising a combination of a first thickening agent which is xanthan gum, a second thickening agent which is carrageenan and/or a polyacrylic acid and a third thickening agent which is a thickening silica, together with a post-foaming agent to expand the composition to a foam.

The presence of carrageenan and/or a polyacrylic acid helps to reduce the stringiness of a post-foaming dentifrice comprising xanthan gum. A thickening silica helps to provide good stand-up when dispensed onto a toothbrush. The reduction of stringiness is particularly beneficial for a post-foaming dentifrice in order to minimise the amount of product drooling when dispensed from a bag in can dispenser.

Additionally it is anticipated that the post-foaming nature of the composition of the present invention provides an extremely efficient release of active ingredients and flavouring agents, even to “hard to reach areas” within the oral cavity.

Suitably the second thickening agent is a polyacrylic acid, examples of which include a carbomer, an acrylate/alkyl acrylate cross polymer or a polycarbophil.

Suitably the second thickening agent is carrageenan.

The viscosity of the composition of the present invention is defined by reference to that of the composition before incorporation of the post-foaming agent, known in the art as the intermediate composition, and is measured using a Brookfield Viscometer at 25° C. using a TF spindle at 20 rpm.

Suitably the viscosity of the intermediate composition is from 100,000 to 240,000 mPas, for example from 120,000 to 220,000 mPas.

Suitably the first thickening agent (xanthan gum) is present in an amount from 0.4 to 1.5%, for example from 0.5 to 1.0% by weight of the total composition.

Suitably the second thickening agent is present in an amount from 0.4 to 1.5%, for example from 0.5 to 1.0% by weight of the total composition

Suitably the third thickening agent (thickening silica) is present in an amount from 3 to 15%, for example from 4 to 10% by weight of the total composition.

Compositions of the present invention may comprise one or more post-foaming agents. The post-foaming agent may be a liquid or liquefiable gas having a boiling point at atmospheric pressure of −5 to 40° C., for example from 15 to 35° C., or from 20 to 30° C. The post-foaming agent may be a liquefiable gas having a vapour pressure of about 0.5-1.0 atmospheres in the temperature range 25-35° C.

Suitable liquid or liquefiable gas post-foaming agents are described in WO-A-01/62212, and include saturated aliphatic hydrocarbons having 4-6 carbon atoms, such as butanes, pentanes and hexanes, halogenated hydrocarbons such as trichlorotrifluoroethane, 1,2-dichloro-1,1,2,2-tetrafluoroethane, or mixtures of at least two thereof. Non-halogenated hydrocarbons such as butane, isobutane, pentane or isopentane and mixtures of at least two thereof may be preferred as halogenated agents have become associated with environmental disadvantages.

An advantage of using a mixture of post-foaming agents is that the vapour pressure of the post-foaming agent may be controlled, e.g. so as to have a vapour pressure of about 0.5-1.0 atmospheres in the temperature range 25-35° C.

Typically 0.5-10% by weight, for example from 1 to 4% by weight of a post-foaming agent is used.

In addition to the thickening silica, compositions of the present invention may comprise a dental abrasive, such as a silica abrasive, or may contain no added abrasive, eg as described in WO 05/027858 (Glaxo Group Ltd).

Examples of suitable silica dental abrasives include those marketed under the following trade names Zeodent, Sident, Sorbosil or Tixosil by Huber, Degussa, Ineos and Rhodia respectively.

Suitably a silica abrasive is present in an amount up to 15% by weight of the total composition, for example from 2 to 10% by weight, and typically at least 4%, for example from 5 to 9% by weight, or from 6 to 8% by weight of the total composition.

Compositions of the present invention may comprise one or more active agents conventionally used in dentifrice compositions, for example a source of fluoride ions, a desensitising agent, an antimicrobial agent, an anti-plaque agent; an anti-calculus agent, a whitening agent, an oral malodour agent or a mixture of at least two thereof. Such agents may be included at levels to provide the desired therapeutic effect.

Suitable sources of fluoride ions for use in the compositions of the present invention include an alkali metal fluoride such as sodium fluoride, an alkali metal monofluorophosphate such a sodium monofluorophosphate, stannous fluoride, or an amine fluoride in an amount to provide from 25 to 3500 pm of fluoride ions, preferably from 100 to 1500 ppm. A typical fluoride source is sodium fluoride, for example the composition may contain 0.1 to 0.5% by weight of sodium fluoride, eg 0.204% by weight (equating to 927 ppm of fluoride ions), 0.2542% by weight (equating to 1150 ppm of fluoride ions) or 0.315% by weight (equating to 1426 ppm of fluoride ions).

Such fluoride ions help promote the remineralisation of teeth and can increase the acid resistance of dental hard tissues for combating caries, dental erosion (ie acid wear) and/or tooth wear.

Compositions of the present invention may be formulated to enhance the uptake of fluoride into the teeth, for example as described in WO2006100071 (Glaxo Group Ltd), the entire contents of which are incorporated herein by reference.

In order to treat dental hypersensitivity, compositions of the present invention may comprise a desensitising agent. Examples of desensitising agents include a tubule blocking agent or a nerve desensitising agent and mixtures thereof, for example as described in WO 02/15809 (Block). Examples of desensitising agents include a strontium salt such as strontium chloride, strontium acetate or strontium nitrate or a potassium salt such as potassium citrate, potassium chloride, potassium bicarbonate, potassium gluconate and especially potassium nitrate.

A desensitising agent such as a potassium salt is generally present between 2 to 8% by weight of the total composition, for example 5% by weight of potassium nitrate may be used.

Compositions of the present invention may comprise a whitening agent, for example selected from a polyphosphate, eg sodium tripolyphosphate (STP) and/or any additional silica abrasive present may have high cleaning properties. STP may be present in an amount from 2 to 15%, for example from 5 to 10% by weight of the total composition. Examples of high cleaning silica abrasives include those marketed as Zeodent 124, Tixosil 63, Sorbosil AC39, Sorbosil AC43 and Sorbosil AC35 and may be present in suitable amounts as hereinbefore described.

Compositions of the present invention may comprise an oral malodour agent, for example a zinc salt such as zinc oxide.

Compositions of the present invention will contain additional formulating agents such as surfactants, humectants, flavouring agents, sweetening agents, opacifying or colouring agents, preservatives and water, selected from those conventionally used in the oral hygiene composition art for such purposes.

Suitable surfactants for use in the present invention include anionic surfactants such as a sodium C₁₀₋₁₈alkyl sulphate, eg sodium lauryl sulphate. Sodium lauryl sulphate is generally considered to be anionic and strongly charged and is useful if high levels of foaming are desired when brushing teeth.

Alternatively zwitterionic, amphoteric and non- or low-ionic surfactants may be used in addition to or instead of an anionic surfactant.

For a dentifrice composition of the present invention comprising a desensitising agent, such as a potassium salt, typical classes of surfactants include amphoteric or mildly ionic surfactants, or mixtures thereof, these being less ionic and less strongly charged than sodium lauryl sulphate. Suitably compositions of the present invention comprising a potassium salt do not contain an anionic surfactant such as a C₁₋₁₈ alkyl sulphate, eg sodium lauryl sulphate, which, whilst it has good detergent and cleaning properties, can cause the formation of an insoluble potassium lauryl sulphate precipitate.

Examples of amphoteric surfactants include, long chain alkyl betaines, such as the product marketed under the tradename ‘Empigen BB’ by Albright & Wilson, long chain alkyl amidoalkyl betaines, such as cocamidopropylbetaine, or low ionic surfactants such as sodium methyl cocoyl taurate, which is marketed under the trade name Adinol CT by Croda, or a mixture of at least two thereof.

Suitably, the surfactant is present in the range 0.1 to 15%, for example from 1 to 10% or from 2 to 5% by weight of the total composition

Suitable humectants for use in compositions of the invention include glycerin, xylitol, sorbitol, propylene glycol or polyethylene glycol, or mixtures of at least two thereof; which humectant may be present in the range from 10 to 80%, for example from 20 to 70% or from 40 to 60% by weight of the total composition.

The compositions according to the present invention may be prepared by admixing the ingredients in the appropriate relative amounts in any order that is convenient and if necessary adjusting the pH to give a final desired value.

Suitably the pH is from 5.5 to 10, eg from 6.5 to 8, and is measured when the composition is slurried with water in a 1:3 weight ratio of the composition to water.

The composition of the present invention is suitable for containing in and dispensing from a so-called bag in can dispenser. For example the composition without the post-foaming agent, known in the art as an intermediate composition is preferably charged into the collapsible envelope of such a dispenser with a dispensing valve, together with the post-foaming agent. Alternatively the composition may be contained in and dispensed from the reverse construction of bag in can dispenser described above.

In such a bag in can dispenser the pressurised propellant in either construction may be a conventional propellant e.g. as disclosed in U.S. Pat. No. 3,541,581. Typical pressurised propellants include compressed air, compressed nitrogen, compressed carbon dioxide, hydrocarbon propellants such as propane, butane, isobutane, isopentane, dimethyl ether, and hydrofluorocarbon propellants of general formula CₙHₓF_{y}, where n is 1 or 2 and x+y equals 2n+2. Such organic propellants may be present in liquid form. Typically a minimum pressure of 1.5 bar may be used in the space between the bag to compress the bag and squeeze the composition out of the bag. For example in this space compressed air or nitrogen may be used at a pressure of typically 4 to 12 bar, or a hydrocarbon propellant at a pressure of typically 1.5 to 5 bar.

The present invention therefore provides a bag in can dispenser containing a dentifrice composition as described herein. Such a dispenser may comprise the composition contained within a collapsible envelope provided with a dispensing valve which can be opened, the envelope being contained within an enclosing can, and with a pressurised propellant between the can and the envelope, which squeezes the dentifrice composition out of the envelope when the valve on the envelope is opened to the environment. Such a dispenser may alternatively comprise an expandable envelope containing a pressurised propellant contained within an enclosing can, with the composition being in the space between the can and the envelope, the can being provided with a dispensing valve which can be opened, and wherein expansion of the propellant within the bag squeezes the composition out of the space when the valve is opened to the environment.

With a composition containing the thickening and abrasive particulate silicas described herein a valve stem bore of 200-500 microns, preferably 400+/−50 microns, is suitable.

Typically such a bag in can dispenser is provided with an actuator device by means of which the valve can be opened and the flow of composition dispensed via a typical actuator nozzle. The dentifrice composition may be dispensed in a generally conventional manner involving opening the valve of the container using the actuator to allow the pressure of the propellant to expel the composition onto a toothbrush as an extruded strip. Ambient temperature or body temperature exposure to the extruded strip causes the post-foaming agent to evaporate so that the composition expands into an expanded foam state.

Suitably the bag in can dispenser is fitted with an actuator that is designed to minimise product drooling from the nozzle of the actuator when is use. An example of such an actuator is described in PCT application, PCT/EP2007/061173, filed on 18 Oct. 2007 claiming priority from UK patent application, GB620943.1, filed on 20 Oct. 2006 in the name of GlaxoSmithKline Consumer Healthcare GmbH and Co KG.

A typical process for making the composition of this invention involves admixing the ingredients, suitably under a vacuum, until a homogeneous mixture is obtained, and adjusting the pH if necessary. The fluid mixture may then be charged, together with a suitable quantity of post-foaming agent, into a suitable collapsible envelope, or the space between the can and expandable envelope of a conventional bag in can dispenser in a conventional manner.

The invention will now be described by way of the following non-limiting examples.

| | Dentifrice Composition | |
|---|---|---|
| | Example 1 | Example 2 |
| Raw Material | % w/w | % w/w |
| Sorbitol 70% solution - humectant | 30.000 | 31.000 |
| Abrasive silica | 8.000 | 6.000 |
| Thickening silica | 8.000 | 9.000 |
| Glycerin - humectant | 20.000 | 20.000 |
| Polyethylene glycol - humectant | 3.000 | 3.000 |
| Sodium lauryl sulphate - surfactant | 2.000 | 0.000 |
| Cocoamidopropylbetaine - surfactant | 0.650 | 1.000 |
| Sodium methyl cocoyl taurate - | 0.000 | 2.000 |
| surfactant | | |
| Xanthan gum - thickener | 0.700 | 0.700 |
| Carrageenan - thickener | 0.900 | 0.700 |
| Potassium Nitrate - active | 0.000 | 5.000 |
| Sodium Fluoride - active | 0.204 | 0.204 |
| Flavour oil | 1.600 | 1.300 |
| Saccharin sodium - sweetener | 0.300 | 0.350 |
| Sodium Hydroxide - pH adjuster | 0.020 | 0.070 |
| Dye | 0.0016 | 0.0003 |
| Water | To 98 | To 98 |
| Isopentane | 2.000 | 2.000 |
| pH (1:3 dilution) | 7.5 | 7.0 |
| Viscosity (of intermediate | 140,000 | 200,000 |
| composition) | mPas | mPas |

The dentifrice composition of Example 1 or 2 is prepared by admixing the ingredients and adjusting the pH with sodium hydroxide to form an intermediate composition. This intermediate composition is charged with isopentane into a collapsible envelope of a conventional bag in can dispenser in a conventional manner. The can is prefilled (before the intermediate composition is put into the bag) with compressed nitrogen to 2.3 bar. The final pressure target when the product is in the bag is 6.3 bar.

1. A post-foaming dentifrice composition having a viscosity of at least 80,000 mPas comprising a combination of a first thickening agent which is xanthan gum, a second thickening agent which is carrageenan and/or a polyacrylic acid and a third thickening agent which is a thickening silica, together with a post-foaming agent to expand the composition to a foam.

2. A composition according to claim 1 wherein the second thickening agent is carrageenan.

3. A composition according to claim 1 having a viscosity from 100,000 to 240,000 mPas.

4. A composition according to claim 1 wherein the first thickening agent is present in an amount from 0.4 to 1.5% by weight of the total composition.

5. A composition according to claim 1 wherein the second thickening agent is present in an amount from 0.4 to 1.5% by weight of the total composition.

6. A composition according to claim 1 wherein the third thickening agent is present in an amount from 3 to 15% by weight of the total composition.

7. A composition according to claim 1 wherein the post-foaming agent is a liquid or a liquefiable gas having a boiling point at atmospheric pressure of −5 to 40° C.

8. A composition according to claim 7 wherein the post-foaming agent is butane, isobutane, pentane or isopentane, or a mixture of at least two thereof.

9. A composition according to claim 1 wherein the post-foaming agent is present in an amount from 0.5 to 10% by weight of the total composition.

10. A composition according to claim 1 comprising a silica dental abrasive.

11. A composition according to claim 1 comprising a source of fluoride ions.

12. A composition according to claim 1 comprising a desensitising agent.

13. A composition according to claim 1 comprising a whitening agent.

14. A composition according to claim 1 comprising an oral malodour agent.

15. A composition according to claim 1 comprising a surfactant.

16. A composition according to claim 1 comprising an anionic surfactant.

17. A composition according to claim 12 comprising an amphoteric or low ion surfactant or a mixture thereof.

18. A bag in can dispenser containing a dentifrice composition according to any one of the preceding claims, comprising the composition contained within a collapsible envelope provided with a dispensing valve which can be opened, the envelope being contained within an enclosing can, and with a pressurised propellant between the can and the envelope, which squeezes the composition out of the envelope when the valve on the envelope is opened to the environment.

19. A bag in can dispenser containing a dentifrice composition according to any one of the preceding claims, comprising an expandable envelope containing a pressurised propellant contained within an enclosing can, with the composition in the space between the can and the envelope, the can being provided with a dispensing valve which can be opened, and wherein expansion of the propellant within the bag squeezes the composition out of the space when the valve is opened to the environment.
